(19) United States
(12) Patent Application Publication
(10) Pub. No.: US 2017/0127977 A1
(43) Pub. Date: May 11, 2017

(54) NON-INVASIVE MONITORING OF PULMONARY CONDITIONS

(71) Applicant: KONINKLIJKE PHILIPS N.V., EINDHOVEN (NL)

(72) Inventors: Mirela Alina WEFFERS-ALBU, Boukoul (NL); Gijs GELEIJNSE, Geldrop (NL); Emile Josephus Carlos KELKBOOM, Eindhoven (NL)

(73) Assignee: KONINKLIJKE PHILIPS N.V., Eindhoven (NL)

(21) Appl. No.: 15/317,424

(22) PCT Filed: Jun. 17, 2015
(86) PCT No.: PCT/EP2015/063605

§ 371 Date: Dec. 9, 2016

(30) Foreign Application Priority Data
Jun. 26, 2014 (EP) 14174213.0

(51) Int. Cl. A61B 5/08; A61B 5/11; A61B 5/113; A61B 5/00

(52) U.S. Cl. CPC A61B 5/0823; A61B 5/7278; A61B 5/1128; A61B 5/113; A61B 5/4815; A61B 5/7275; A61B 5/0077; A61B 2562/0219

(57) ABSTRACT

A method for non-invasively monitoring a status of a user with a pulmonary condition comprises obtaining first video data of the user during a first test period; analyzing the obtained first video data to determine a first respiratory signal for the user; detecting any cough events present in the first respiratory signal; obtaining second video data of the user during a second, later, test period; analyzing the obtained second video data to determine a second respiratory signal for the user; detecting any cough events present in the second respiratory signal; determining a respiratory status of the user based on the results of the detecting and outputting a signal containing information about the respiratory status of the user.

### TECHNICAL FIELD OF THE INVENTION

The invention relates to monitoring the status of a user with a health condition. More specifically, the invention relates to a method, apparatus and system for the non-invasive monitoring of the status of a user with a pulmonary condition.

### BACKGROUND TO THE INVENTION

Research has shown that deterioration in the status of a subject suffering from a pulmonary condition (such as, for example, asthma, chronic obstructive pulmonary disease (COPD), emphysema, cystic fibrosis, etc.) is characterized by a combination of aspects. Respiration deficiency causes dyspnea (shortness of breath) and coughing. Indeed, increased dyspnea and increased sputum purulence and/or volume (which leads to increased coughing) are generally agreed to be the most distinct or cardinal symptoms of an exacerbation of a pulmonary disease (see, e.g., G C Donaldson and J A Wedzicha, “COPD exacerbations—1: Epidemiology”, Thorax 2006; 61:164-168. doi: 10.1136/thx.2005.041806; Anthonisen N R, Manfreda J, Warren C P, et. al. “Antibiotic therapy in exacerbations of chronic obstructive pulmonary disease”, Ann. Intern. Med. 1987; 106:196-204). Other aspects which characterize deterioration are poor quality of sleep and fatigue/lack of vitality (see, e.g., American Thoracic Society, “Dyspnea—Mechanisms, Assessment, and Management: A Consensus Statement”, American Journal of Respiratory and Critical Care Medicine, vol. 159, 1999; G C Donaldson and J A Wedzicha, “COPD exacerbations−1: Epidemiology”, Thorax 2006; 61:164-168. doi: 10.1136/thx.2005.041806; Collop N., “Sleep and sleep disorders in chronic obstructive pulmonary disease”, Respiration, 2010; 80(1):78-86. doi: 10.1159/000258676; McSharry D G et. al., “Sleep quality in chronic obstructive pulmonary disease”, Respirology, 2012 October; 17(7):1119-24. doi: 10.1111/j.1440-1843.2012.02217.x)

Current state of the art techniques for monitoring the status of subjects with pulmonary conditions use telemonitoring systems such as the AMICA project in Spain, COPD Home Monitoring Solutions by Zydacron Telecare gmbh, the Telehealth Solution by Care Cycle Solutions, and the COPD telemonitoring service provided by NHS Lothian in the UK, as well as telemedicine services for the purpose of managing exacerbations, such as those proposed in Maarten Van Der Heij den, et. al. “Managing COPD exacerbations with telemedicine”, Artificial Intelligence in Medicine, Springer Berlin Heidelberg, 2011, 169-178. Telemonitoring involves remotely monitoring patients who are not at the same location as the health care provider. In such systems a subject is provided with number of monitoring devices at their home, which they must use to measure physiological parameters (such as, for example, blood pressure, heart rate, weight, blood glucose, etc.). The results obtained by the monitoring devices are sent, e.g. via telephone or the internet, to the health care provider.

Telemonitoring systems are generally received well by users, both on the patient side and the caregiver side. However; there are several disadvantages associated with existing systems. In particular, many systems are not easy to install or for patients to use, most systems require significant input from health care professionals for their use and management, and many are expensive due to the specialist hardware required (e.g. medical monitoring devices, sensors, communications equipment, etc.).

There is therefore a need for a low-cost, easy-to-use monitoring system that can provide a reliable assessment of the status of a user with a pulmonary condition. Such a system could be used in place of or alongside a telemonitoring system, for assessing the current health status of the user and the likelihood of their condition worsening, for detecting worsening of the pulmonary condition, and/or for monitoring improvements in the user's status as a result of receiving treatment. Such a system could provide a caregiver with early insights into the patient status and thereby allow timely interventions, before the condition takes a critical/acute form.

### SUMMARY OF THE INVENTION

According to a first aspect of the invention, there is provided a method for non-invasively monitoring a status of a user with a pulmonary condition, the method comprising,
(a) providing a processing unit:
(b) obtaining first video data of the user during a first test period;
(c) analyzing the obtained first video data to determine a first respiratory signal for the user;
(d) detecting any cough events which are present in the first respiratory signal, said cough events being detected from peaks of the first respiratory signal;
(e) obtaining second video data of the user during a second, later, test period;
(f) analyzing the obtained second video data to determine a second respiratory signal for the user;
(g) detecting any cough events which are present in the second respiratory signal, said cough events being detected from peaks of the second respiratory signal;
(h) determining a respiratory status of the user based on the result of the detecting in of steps (d) and (g); and
(i) outputting a signal containing information about the respiratory status of the user, wherein at least steps (c), (d) and (f)-(i) are performed by the provided processing unit.

Embodiments of the current invention provide a way of unobtrusively monitoring the value trends of parameters characteristic of the deterioration of the status of a patient with a pulmonary condition. These trends are used to assess and estimate the current status, together with the likelihood of pulmonary condition worsening. This information provides the user's caregiver with early insights into the user's status and thereby allows for timely interventions to prevent significant negative developments.

Advantageously, the invention can be implemented, in certain embodiments, as an application for a portable electronic device such as a smartphone or tablet computer, which uses the built-in capabilities of the device to periodically measure physiological parameters of the user and to inform caregivers of the user's status. Such embodiments are significantly less expensive than conventional telemonitoring systems, since they do not require specialist hardware, and are also easier to install and use and are more convenient for the user. Furthermore, embodiments of the invention do not require trained health professionals in order to be operated, so can lessen the burden on healthcare providers.

In some embodiments the method additionally comprises obtaining further video data between obtaining the first video data and obtaining the second video data. In such embodiments one or more further respiratory signals are determined based on the further video data, and cough events are also detected in the one or more further respiratory signals. In such embodiments the determining of the respiratory status is additionally based on the results of detecting cough events in the one or more further respiratory signals.

In particular embodiments of the invention step (d) comprises determining the number of cough events present in the first respiratory signal and step (g) comprises determining the number of cough events present in the second respiratory signal. In some such embodiments step (h) comprises:
comparing the results of steps (d) and (g) with an upper threshold and a lower threshold; and if the number of detected cough events during the predefined time frame is less than the lower threshold, determining the respiratory status of the user as a first risk level;
if the number of detected cough events during the predefined time frame is greater than or equal to the lower threshold but less than the upper threshold, determining the respiratory status of the user as a second risk level; and
if the number of detected cough events during the predefined time frame is greater than or equal to the upper threshold, determining the respiratory status of the user as a third risk level.

In some embodiments the first risk level is a low risk level, the second risk level is a medium risk level and the third risk level is a high risk level. In some embodiments the first, second and third risk levels are associated with first, second and third predefined colors, which may be used, for example, in a message to a caregiver. This advantageously enables the caregiver to very quickly ascertain the current risk level of a user on receipt of such a message.

In preferred embodiments the method further comprises detecting and analyzing features associated with dyspnea in the first respiratory signal and in the second respiratory signal. In some such embodiments the step of detecting and analyzing features associated with dyspnea in the first respiratory signal and in the second respiratory signal comprises:
calculating values of the mean respiration rate and mean respiration amplitude for each respiratory signal;
comparing the calculated mean respiration rate values to one or more predefined thresholds; and
analyzing the calculated mean respiration rate values and mean respiration amplitude values to identify trends in the mean respiration rate and mean respiration amplitude.

Detecting and analyzing features associated with dyspnea as well as detecting cough events can advantageously improve the accuracy of assessments of current user respiratory status and/or predictions of future user respiratory status generated based by the method. This in turn can improve the accuracy of assessments of current status of the user's pulmonary condition and/or predictions of future status of the user's pulmonary condition generated based by the method.

In preferred such embodiments, the method additionally comprises obtaining further video data between obtaining the first video data and obtaining the second video data, determining one or more further respiratory signals based on the further video data, and detecting and analyzing features associated with dyspnea in the one or more further respiratory signals. In particularly preferred embodiments video data is obtained once per day, and the second video data is obtained seven days after the first video data is obtained. In such embodiments the further video data comprises five sets of video data and five further respiratory signals are determined.

In some embodiments the respiratory status is determined based additionally on the results of the detecting and analyzing of features associated with dyspnea.

In alternative embodiments, the method further comprises determining a dyspnea status of the user based on the results of the detecting and analyzing of features associated with dyspnea. In such alternative embodiments the signal output in step (i) additionally contains information about the dyspnea status of the user. In some such embodiments the method further comprises determining mean respiration rate values which exceed the one or more predefined thresholds to be indications of dyspnea. In some such embodiments determining a dyspnea status of the user comprises:
if none of the calculated mean respiration rate values are determined to be indications of dyspnea, and no sustained trends of decreasing respiration amplitude and/or increasing respiration rate are identified, determining the dyspnea status of the user as a first risk level;
if none of the calculated mean respiration rate values are determined to be indications of dyspnea and at least one sustained trend of decreasing respiration amplitude and/or increasing respiration rate is identified, determining the dyspnea status of the user as a second risk level; and
if the number of mean respiration rate values which are determined to be indications of dyspnea during a predefined time frame is greater than or equal to a predefined dyspnea indication threshold, determining the dyspnea status of the user as a third risk level.

In some embodiments the first risk level is a low risk level, the second risk level is a medium risk level and the third risk level is a high risk level. In some embodiments the first, second and third risk levels are associated with first, second and third predefined colors.

In some embodiments, the method further comprises sending or displaying a reminder message to the user, if the first and/or second video data has not been obtained by a predefined time. Such embodiments can advantageously improve patient compliance with a monitoring regime.

In some embodiments, the signal output in step (i) is arranged to cause a message containing the information contained in the signal to be sent to an electronic device associated with a caregiver. In some such embodiments the message comprises an SMS message. In other such embodiments the message comprises an e-mail message.

In some embodiments the method further comprises:
obtaining first waking activity measurements of the activity of the user during a third test period;
obtaining second waking activity measurements of the activity of the user during a fourth test period;
analyzing the first and second waking activity measurements to detect trends in the waking activity levels of the user; and
determining a waking activity status of the user based on the detected waking activity trends.

In such embodiments the signal output in step (i) additionally contains information about the waking activity status of the user. Waking activity levels generally reflect the degree of fatigue/lack of vitality experience by a user. Since increased fatigue/lack of vitality is one characteristic that is associated with the worsening of a pulmonary condition, such embodiments can potentially generate more accurate assessments of the current status of the user's pulmonary condition and/or more accurate predictions of the future status of the user's pulmonary condition.

In some embodiments the method further comprises:
obtaining first sleep motion measurements of the activity of the user during a fifth test period;
obtaining second sleep motion measurements of the activity of the user during a sixth test period;
analyzing the first and second sleep motion measurements to detect trends in the sleep quality of the user; and
determining a sleep quality status of the user based on the detected sleep quality trends.

In such embodiments the signal output in step (i) additionally contains information about the sleep quality status of the user. Since decreased sleep quality is one characteristic that is associated with the worsening of a pulmonary condition, such embodiments can potentially generate more accurate assessments of the current status of the user's pulmonary condition and/or more accurate predictions of the future status of the user's pulmonary condition.

According to a second aspect of the invention, there is provided a portable device for non-invasively monitoring a status of a user with a pulmonary condition. The device comprises:
a processing unit having a camera input for receiving video data of the user obtained by a camera, wherein the processing unit is configured to perform at least steps (c), (d) and (f)-(i) of the method of the first aspect.

In preferred embodiments the device further comprises a camera for obtaining video data of the user, connected to the camera input. In such embodiments the processing unit is configured to perform the method of the first aspect, wherein the processing unit is configured to perform step (b) of the method of the first aspect by triggering the camera to capture video data during a first time period, and to perform step (e) of the method of any of claims 1-9 by triggering the camera to capture video data during a second, later, time period.

In preferred embodiments the portable device further comprises a communications interface for sending and/or receiving data to/from another device. Such embodiments advantageously allow the portable device to, for example, receive measurements taken by additional sensors, to send messages to a caregiver, send data to a remote server, etc. In preferred embodiments the portable device comprises one of: a smartphone, a tablet computer, a laptop computer, a personal digital assistant, a digital camera.

In some embodiments, the processing unit of the portable device is further configured to determine the respiratory status based additionally on the results of the detecting and analyzing of features associated with dyspnea.

In alternative embodiments, the processing unit of the portable device is further configured for determining a dyspnea status of the user based on the results of the detecting and analyzing of features associated with dyspnea. In such alternative embodiments the signal output in step (i) additionally contains information about the dyspnea status of the user. In some such embodiments the method further comprises determining mean respiration rate values which exceed the one or more predefined thresholds to be indications of dyspnea. In some such embodiments determining a dyspnea status of the user comprises:
if none of the calculated mean respiration rate values are determined to be indications of dyspnea, and no sustained trends of decreasing respiration amplitude and/or increasing respiration rate are identified, determining the dyspnea status of the user as a first risk level;
if none of the calculated mean respiration rate values are determined to be indications of dyspnea and at least one sustained trend of decreasing respiration amplitude and/or increasing respiration rate is identified, determining the dyspnea status of the user as a second risk level; and
if the number of mean respiration rate values which are determined to be indications of dyspnea during a predefined time frame is greater than or equal to a predefined dyspnea indication threshold, determining the dyspnea status of the user as a third risk level.

In some embodiments the first risk level is a low risk level, the second risk level is a medium risk level and the third risk level is a high risk level. In some embodiments the first, second and third risk levels are associated with first, second and third predefined colors.

According to a third aspect of the invention there is provided a system for non-invasively monitoring a status of a user with a pulmonary condition. The system comprises:
a portable device according to the second aspect, configured to receive activity measurements from a sensor; and
one or more sensors for measuring activity of the user, configured to send activity measurements to the portable device;
   wherein the processing unit is configured to perform embodiments of the method of the first aspect which comprise obtaining and analyzing sleep quality measurements, and/or which comprise obtaining and analyzing waking activity measurements. The processing unit is configured to perform the steps of obtaining sleep motion measurements and obtaining waking activity measurements by receiving activity measurements from the one or more sensors.

In preferred embodiments the one or more sensors comprise an activity actigraph and/or a sleep actigraph. In some embodiments the one or more sensors comprise an accelerometer. In some embodiments the one or more sensors comprise a gyroscope.

According to a fourth aspect of the invention there is provided a computer program product, comprising computer readable code embodied therein, the computer readable code being configured such that, on execution by a suitable computer or processing unit, the computer or processing unit performs the method of the first aspect.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is an illustration of a system for monitoring the status of a user with a pulmonary condition according to a first specific embodiment of the invention;

FIG. 2 is a flow chart illustrating a method for monitoring the status of a user with a pulmonary condition according to a general embodiment of the invention;

FIG. 3a shows a first example of a respiration pattern which includes coughing events;

FIG. 3b shows a second example of a respiration pattern which includes coughing events;

FIG. 4 is a graph showing a normal respiration signal and a respiration signal with dyspnea; and

FIG. 5 is a flow chart illustrating additional method steps for monitoring the status of a user with a pulmonary condition based on activity levels as well as respiratory signals, according to second specific embodiment of the invention.

### DETAILED DESCRIPTION OF THE EMBODIMENTS

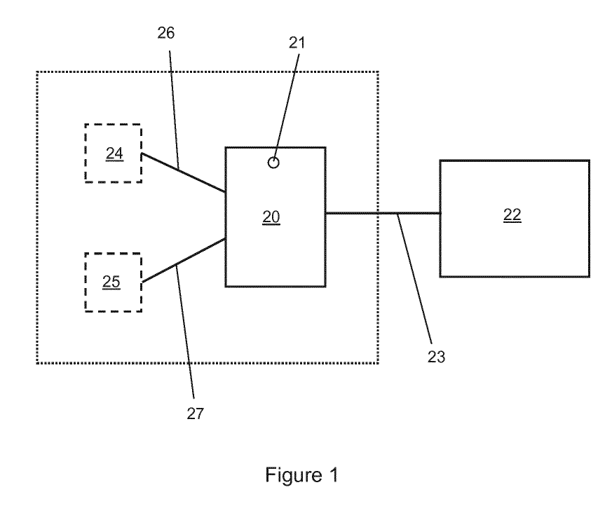

FIG. 1 shows a system for monitoring the status of a user with a pulmonary condition according to a first embodiment of the invention. The system includes a portable electronic device 20 which has a camera 21 and a processing unit. In preferred embodiments the portable electronic device is a smartphone or a tablet computer. The camera 21 is able to acquire video data over periods lasting several minutes. The processing unit is configured to analyze video data of the user to determine a respiratory signal and to detect any cough events present in the respiratory signal. In some embodiments the processing unit is also configured to detect any indications of dyspnea present in the respiratory signal. The processing unit is further configured to determine a respiratory status of the user based on the results of detecting cough events (and, optionally, indications of dyspnea) and to output a signal based on the determined status. In preferred embodiments the signal causes a message (e.g. an SMS text message or an e-mail) to be generated and sent to a caregiver. In some embodiments the processing unit is configured to receive measurements from external sensor devices and to perform analysis on such measurements.

For the present invention, the term portable shall be interpreted as qualifying a device in such way that it is easily carried or moved without external aid by a normal user. As mentioned above, a smarphone or a tablet are non limiting examples of portable devices.

In preferred embodiments the portable electronic device 20 includes a communications interface to enable it to send and/or receive data to/from one or more other electronic devices. The communications interface can utilize any suitable communications technology known in the art, such as Bluetooth, SMS messaging, e-mail etc. In preferred embodiments the communications interface is configured to utilize more than one such communications technology.

The system optionally also includes a first additional sensor 24. In preferred embodiments the first additional sensor 24 is a sleep actigraph, which is an accelerometer configured to be worn on the user's wrist during the night. The system optionally also includes a second additional sensor 25. In preferred embodiments the second additional sensor 25 is an activity actigraph, which is an accelerometer configured to be worn by the user, e.g. on their hip, during the day. The dotted line enclosing components 20, 24 and 25 in FIG. 1 indicates that these components form the patient side of the system (i.e. they will generally be located on or proximal to the subject having the pulmonary condition during use of the system).

The first additional sensor 24 (if present) is configured to send data to the communications interface of the portable electronic device 20 via a first communications link 26. The second additional sensor 25 (if present) is configured to send data to the communications interface of the portable electronic device 20 via a second communications link 27. In preferred embodiments the communications links 26 and 27 are wireless communications links, for example utilizing Bluetooth, infrared or WiFi communications protocols. It will be appreciated, however; that wired links could also be used for one or both of the communications links 26 and 27.

The processing unit of the portable electronic device 20 is configured to receive data from the first additional sensor 24 and/or the second additional sensor 25 and to analyze the received data. In embodiments where the first additional sensor 24 is a sleep actigraph the processing unit is configured to determine a sleep quality value of the user based on data received from the first additional sensor 24. In embodiments where the second additional sensor 25 is an activity actigraph the processing unit is configured to determine a waking activity level of the user based on data received from the second additional sensor 25. In such embodiments the processing unit is further configured to determine a sleep status of the user and/or an activity status of the user and to output a signal based on the sleep status and/or the activity status as well as on the determined respiratory status.

In some embodiments the portable electronic device 20 is configured to use its communications interface to send data to a remote server, for example a server of a healthcare provider, via a communications link 23. In preferred embodiments communications link 23 utilizes a telephone network, or, where available, an internet connection. In some embodiments the portable electronic device can also receive data via the communications link 23.

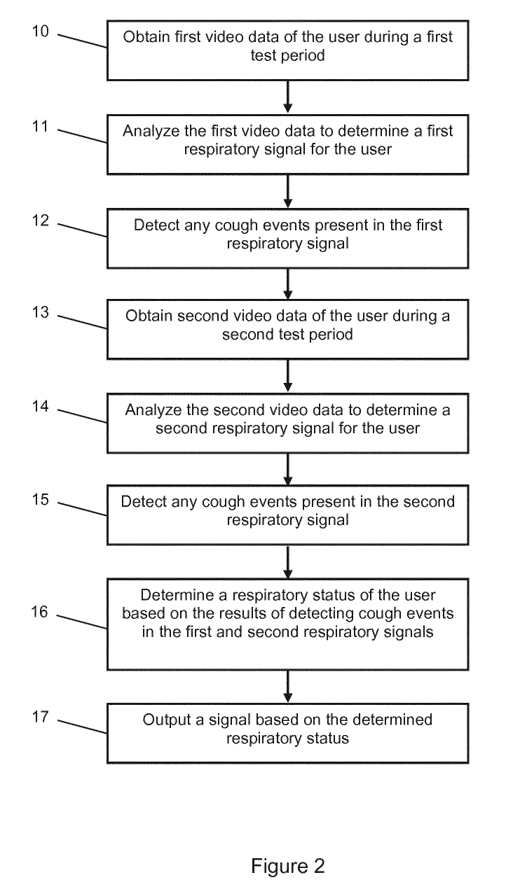

FIG. 2 shows a method for monitoring the status of a user with a pulmonary condition according to an embodiment of the invention.

In step 10, first video data of the user during a first test period is received or obtained, for example from the camera 21. To obtain this data, the user records them self sitting still in the camera's field of view for the duration of a first test period (preferably at least a few minutes). The portable electronic device should be arranged such that the head and torso of the user are within the image. This is very easy to achieve if the portable electronic device 20 is a smartphone or a tablet computer because such devices typically have a front-facing camera which allows the user to look at the screen of the device (which can be made to show the image captured by the camera) whilst being imaged by the camera. Preferably the duration of the first test period (and therefore of the recording) is in the range of 2-10 minutes. In particularly preferred embodiments the duration of the first test period is 10 minutes. In some preferred embodiments, if the user has not obtained the first video data by a certain time (6 pm, say, if daily tests are required) a reminder is generated, for example by the portable electronic device 20, and is displayed by the device or sent to the user (e.g. by SMS or e-mail).

In step 11, the first video data is analyzed, for example by the processing unit of the portable electronic device 20, to determine a first respiratory signal. The first respiratory signal is extracted from the first video data by analyzing motion vectors in the first video data. Techniques suitable for performing the extraction are known in the art. This method of acquiring a respiratory signal has the advantages of being both unobtrusive (since it may be performed at a time and place convenient for the user, and does not involve recording any of their personal activities or interactions) and computationally efficient. Determining a respiratory signal from audio data, for example, is significantly more complicated because the audio data will contain background noise that needs to be filtered before a reliable respiratory signal can be obtained. Since relatively few processing resources are required to determine a respiratory signal from video data obtained in the manner described above, the analysis can easily be performed by the processing unit of a conventional portable electronic device such as a smartphone.

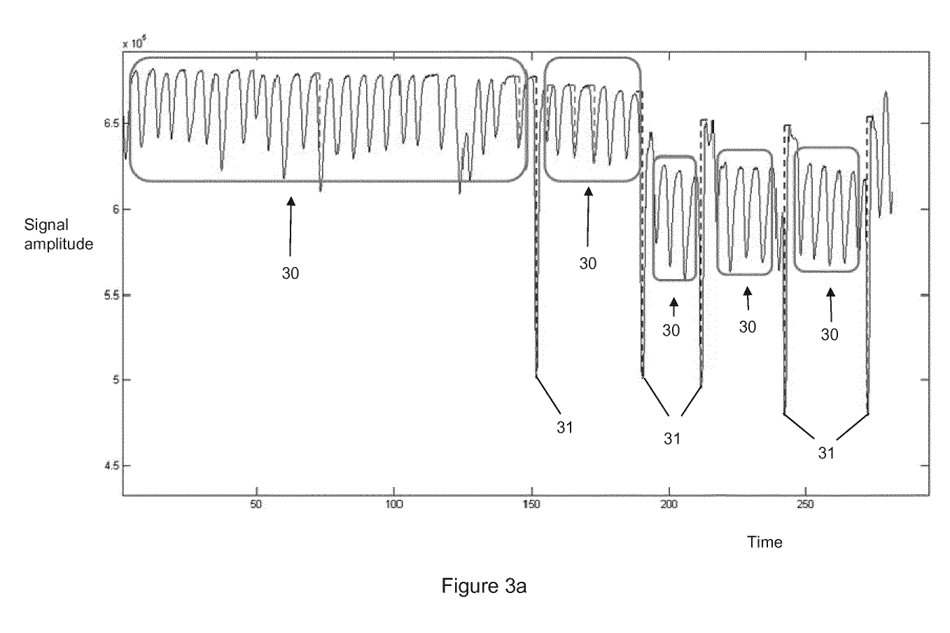

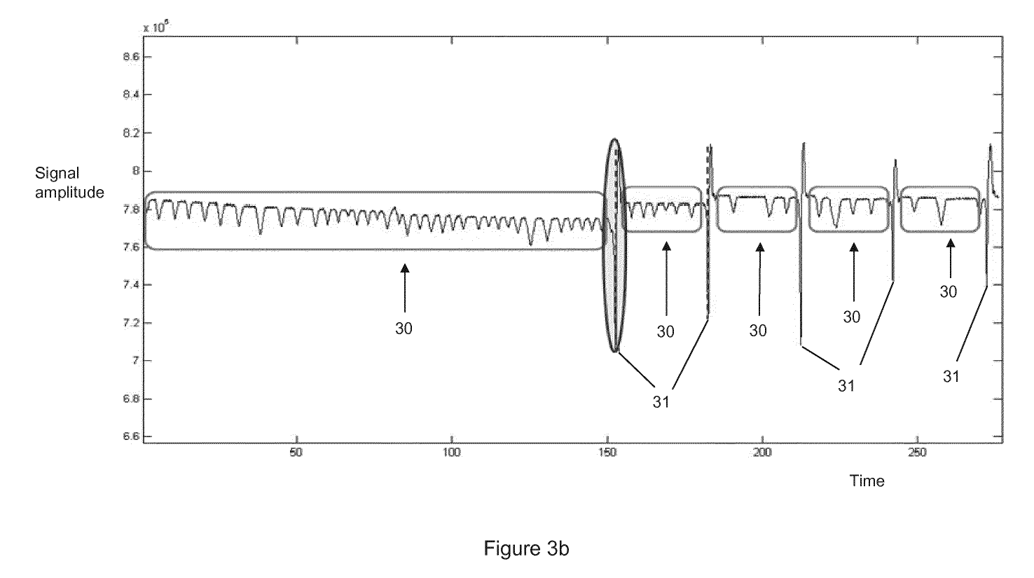

If the user coughed during the obtaining of the first video data, then this will be represented in the first respiratory signal. In step 12, any cough events present in the respiratory signal are detected, e.g. by the processing unit of the portable electronic device 20. Cough events are detected by detecting peaks in the respiratory signal and comparing the difference between the signal amplitude values of each adjacent high peak and low peak. FIGS. 3a and 3b show two different examples of respiratory signals, which each include periods of normal (resting) respiration 30 and cough events 31. In both figures the x-axis shows the signal amplitude and the y-axis shows time. If the difference between the amplitude of each peak in a pair of adjacent peaks is greater than a predefined threshold, the lower amplitude peak is determined to indicate the time stamp of a cough event 31. In some embodiments the predefined threshold is user-specific (i.e. its value is chosen based on data relating to the user in question). In some embodiments the value of the predefined threshold may differ for each given respiratory signal. In some such embodiments the predefined threshold is defined to be a particular fraction of the average amplitude of the inhale-exhale cycle for a given respiratory signal. In some preferred embodiments the predefined threshold is defined to be 0.5×the average amplitude of the inhale-exhale cycle for a given respiratory signal. In alternative embodiments the predefined threshold may be the same for each different respiratory signal. In some such embodiments the value of the predefined threshold is chosen based on historical data (e.g. historical respiratory signal data) for the user. In preferred embodiments step 12 involves determining the number of cough events present in the first respiratory signal (which may, of course, be zero).

In step 13, second video data of the user during a second test period is received or obtained, in the same manner as the first video data. Preferably the second test period has the same duration as the first test period. Step 13 is performed after step 10, such that the first and second test periods are spaced apart in time. Preferably step 13 is performed at least one day after step 10. Preferably step 13 is performed not longer than seven days after step 10. It will be appreciated that the precise length of time which elapses between the performance of steps 10 and 13 is not crucial to the functioning of the invention. Indeed, in preferred embodiments the user may vary the times at which they obtain the first and second video data to enable them to perform these steps at times which are convenient for them. In some embodiments the user is requested (for example by means of a message generated by the portable electronic device 20) to perform steps 10 and/or 13 within a specified time window. Preferably the times at which step 10 and step 13 are performed are recorded, for example by the processing unit of the portable electronic device 20. It will be appreciated that steps 10 and 13 need not represent consecutive acquisitions of video data by the user. For example, the user may obtain video data in the manner of steps 10 and 13 once every day but the first and second video data is spaced apart by more than one day. For example, in an embodiment the second video data is the most recently obtained video data, and the first video data is that which was obtained three days previously. In preferred embodiments, the second video data is the most recently obtained video data, and the first video data is that which was obtained seven days previously.

In step 14 the second video data is analyzed to determine a second respiratory signal, in the same manner as the first video data is analyzed to determine the first respiratory signal. In step 15 any cough events present in the second respiratory signal are detected, using the same techniques as used in step 12.

In step 16 the results of steps 12 and 15 are used, for example by the processing unit of the portable electronic device 20, to determine a respiratory status of the user. If additional video data and associated respiratory signal(s) has also been obtained and analyzed in the time between the acquisition of the first video data and the acquisition of the second video data, then in some embodiments the results of cough detection for this additional signal(s) is also used in the determination of the respiratory status.

The respiratory status of the user is determined as follows. Upper and lower thresholds for the number of cough events detected during a predefined time frame are defined. For example, in a specific embodiment in which video data is obtained and analyzed once each day, the lower threshold is defined to be one detected cough event in the week leading up to (i.e. ending with) the current video data and the upper threshold is defined to be two detected cough events in the same period. If the number of detected cough events is less than the lower threshold (i.e. in the above example, if no cough events are detected over the week) and the current respiratory status of the user is determined to be low risk. In some embodiments the low risk status is represented by the color green. If the number of detected cough events greater than or equal to the lower threshold but less than the upper threshold (i.e. in the above example, if one cough event is detected over the week), the current respiratory status of the user is determined to be medium risk. In some embodiments the medium risk status is represented by the color yellow. A medium risk respiratory status indicates to a caregiver that the user should be approached for a consultation, for example to assess and manage the risk of the user developing an inter-current condition (e.g. flu, pneumonia) that could lead to an exacerbation. If the number of detected cough events is greater than or equal to the upper threshold (i.e. in the above example, if more than one cough event is detected over the week), the current respiratory status of the user is determined to be high risk. In some embodiments the high risk status is represented by the color red. A high risk respiratory status indicates to a caregiver that the user should be provided with a stronger treatment to manage their pulmonary condition.

In step 17, a signal is output, for example by the processing unit of the portable electronic device 20, based on the results of step 16. In preferred embodiments the signal causes the portable electronic device 20 to generate a message containing information about the current respiratory status of the user as determined in step 16. This message may be sent to a caregiver, and/or displayed to the user. Preferably the portable electronic device 20 is configured to generate and send such messages with a predefined frequency, which may, but need not, be equal to the frequency with which the signal is output. For example, in some embodiments a signal is output every time new video data is obtained an analyzed (e.g. daily), but the portable electronic device is configured to send a message to a caregiver weekly. In this case the message generated contains information relating to all of the signals output during the preceding week.

It will be appreciated that steps 10-17 need not be performed in the exact order shown in FIG. 2. For example, in some embodiments steps 11 and 12 are performed after step 13. In some embodiments steps 11 and 12 are performed concurrently with steps 14 and 15.

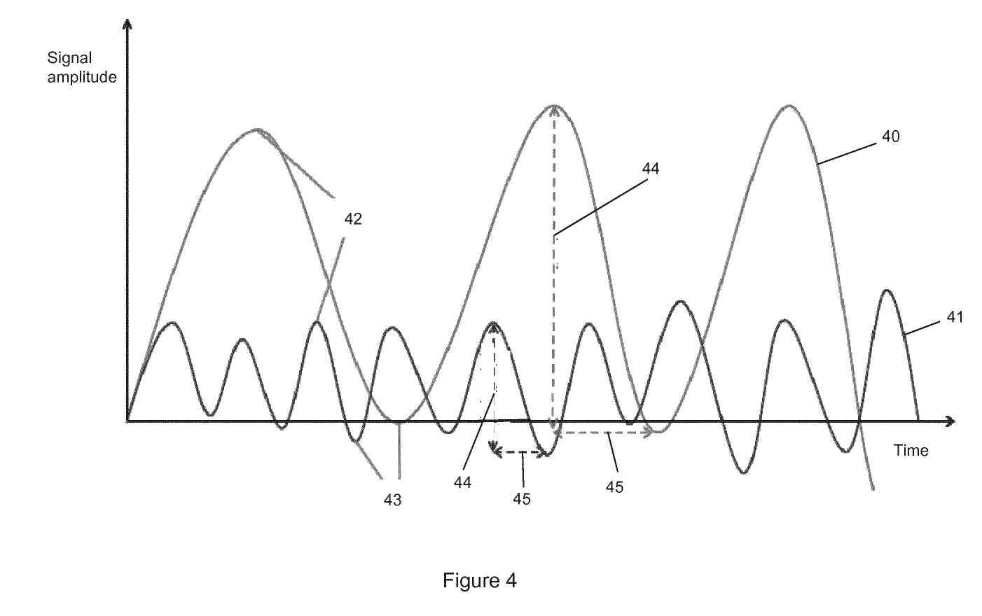

If the user experienced dyspnea during the obtaining of video data, then this will be represented in the respiratory signal determined from that video data. FIG. 4 shows a normal resting respiration signal 40 and a resting respiration signal where dyspnea is present 41. High peaks 42 in the signal represent exhalations and low peaks 43 represent inhalations. Dyspnea is characterized by shallow and rapid breathing. Shallow breathing is represented in the respiration signal by an inhale-exhale amplitude 44 which is significantly decreased compared with normal breathing. Rapid breathing is represented in the respiration signal by a high respiration rate (number of inhale-exhale cycles/min) compared with normal breathing. A normal resting respiration rate is typically within 10-18 inhale-exhale cycles/min. Resting respiration rates that are higher than 18 cycles/min are outside healthy bounds. A high respiration rate also implies low inhale-exhale duration 45. A respiratory signal in which the mean inhale-exhale duration is significantly lower than in normal breathing is therefore indicative of dyspnea.

In some embodiments the method involves detecting any indications of dyspnea which are present in the first and second respiratory signals. The mean respiration rate and mean respiration amplitude (and in some embodiments also the mean inhale-exhale duration) are calculated for a given respiratory signal. The mean respiration rate values are compared to predefined bounds which correspond to the range of normal variability for a healthy subject. Any calculated mean respiration rate value which is outside the bounds is determined to be an indication of dyspnea. For example, in one specific embodiment a lower bound for the mean respiration rate is defined to be 10 inhale-exhale cycles per minute and an upper bound for the mean respiration rate is defined to be 18 inhale-exhale cycles per minute. In this specific embodiment any mean respiration rate value which is less than 10 or greater than 18 inhale-exhale cycles per minute is determined to be abnormal and therefore an indication of dyspnea. In some alternative embodiments a single threshold can be used instead of bounds. For example, in a specific embodiment a respiration rate threshold is defined to be 18 inhale-exhale cycles per minute. Mean respiration rate values less than or equal to this threshold are considered normal, whilst mean respiration rate values greater than this threshold are determined to be indications of dyspnea. In preferred embodiments, the calculated mean values for each given respiratory signal are stored, for example in a memory of the portable electronic device 20.

In preferred embodiments, in addition to characterizing each calculated mean respiration rate value as either normal or an indication of dyspnea, calculated mean respiration rate values and mean respiration amplitude values which span a predefined time frame (i.e. mean values which are calculated based on video data acquired during this time frame) are analyzed to identify trends in these values, using any suitable trend analysis techniques known in the art. In a preferred embodiment in which new video data is acquired daily, the predefined time frame is defined to be the week leading up to (and including) the acquisition of the latest video data, so that seven sets of mean values are used in the trend analysis. In some embodiments a sustained trend of decreasing mean inhale-exhale amplitude is determined to be an indication of dyspnea. In some embodiments a substance trend of increasing mean respiration rate is determined to be an indication of dyspnea.

The detected indications of dyspnea from the predefined time frame are used to determine a dyspnea status of the user. In one embodiment, if no indications of dyspnea are detected during the predefined time frame (i.e. none of the calculated mean respiration rate values are determined to violate normal bounds/thresholds, and no sustained trends of decreasing respiration amplitude and/or increasing respiration rate are identified), then the user is determined to have a low risk (green) dyspnea status. If none of the calculated mean respiration rate values from the predefined time frame are abnormal but a sustained trend of decreasing inhale-exhale amplitude and/or a sustained trend of increasing respiration rate is identified, the user is determined to have a medium risk (yellow) dyspnea status. A medium risk dyspnea status indicates to a caregiver that the patient is at risk of developing dyspnea. If at least one of the calculated mean respiration rate values from the predefined time frame is determined to violate the bounds/threshold, the user is determined to have a high risk (red) dyspnea status. A high risk dyspnea status indicates to a caregiver that dyspnea onset has occurred.

In embodiments in which dyspnea is monitored as well as cough events, a signal is output based on the both the determined respiratory status and on the determined dyspnea status. In such embodiments the signal is as discussed above in relation to step 17 of FIG. 2, except that it also contains information about the current dyspnea status of the user.

Further evolution of trends in the mean values can be revealed by continued acquisition and analysis of video data and associated respiratory signals. The method can therefore be used to detect negative or positive progressions of dyspnea for the purposes of managing the user's pulmonary condition, keeping symptoms under control, and preventing unplanned hospitalizations.

In some embodiments in which indications of dyspnea are detected in addition to cough events, the detected cough events and the detected indications of dyspnea are used to determine an overall respiratory status of the user, rather than a respiratory status (based only on cough events) and a separate dyspnea status. In a specific embodiment, In one embodiment, if no indications of dyspnea are detected during the predefined time frame, and the number of coughing events detected during the predefined time frame is less than a predefined lower threshold, then the user is determined to have a low risk (green) overall respiratory status. If none of the calculated mean respiration rate values from the predefined time frame are abnormal but a sustained trend of decreasing inhale-exhale amplitude and/or a sustained trend of increasing respiration rate is identified and/or the number of coughing events detected during the predefined time frame is greater than or equal to the lower threshold and less than a predefined upper threshold, the user is determined to have a medium risk (yellow) overall respiratory status. If at least one of the calculated mean respiration rate values from the predefined time frame is determined to violate the bounds/threshold, or the number of coughing events detected during the predefined time frame is greater than or equal to the upper threshold, the user is determined to have a high risk (red) overall respiratory status.

Thus, the method in FIG. 2 can accurately assess the health status of a patient with a pulmonary condition and thereby inform caregivers of this status. The method can indicate when deteriorating trends occur, in order to trigger timely interventions for the purpose of disease management and preventing critical worsening. Advantageously, the method can be performed using only a readily available portable electronic device such as a smartphone or a tablet computer, making it convenient, easy to use and inexpensive. Furthermore, it readily allows for the incorporation of additional data from generally available sensor devices such as sleep and activity actigraphs, which can be used to improve the depth and accuracy of the assessment.

Indeed, in some embodiments the method involves measuring activity of the user, for example with the additional sensor 24 and/or the additional sensor 25. If the activity of the user is measured whilst they are asleep it is indicative of sleep quality. If the activity of the user is measured during the course of the user's normal daily routine (which need not occur during the day, e.g. if the user is a shift worker) it is indicative of waking activity levels and can therefore reveal fatigue/lack of vitality. In preferred embodiments the activity of the user is measured both during sleep and during their daily routine, although it will be appreciated that in other embodiments the method can involve measuring only sleep motion or only waking activity.

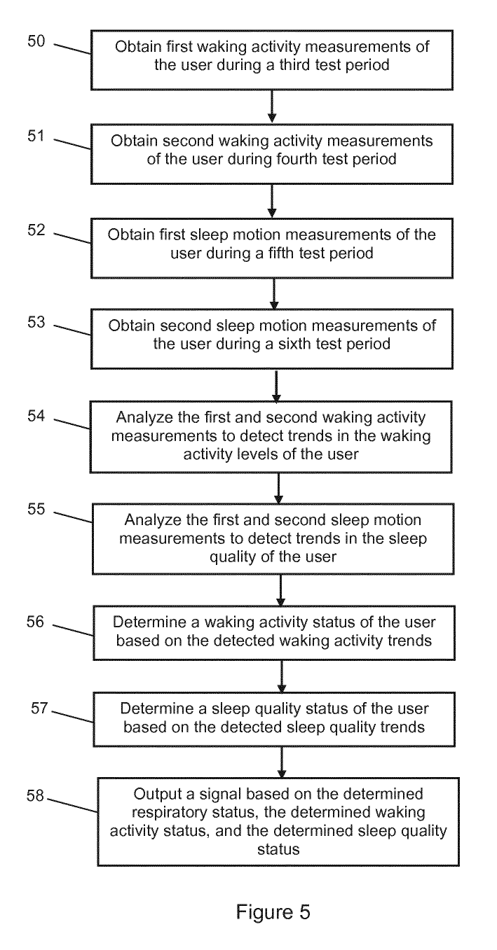

An embodiment in which both sleep motion and waking activity are measured will now be described with reference to FIG. 5. In this embodiment a respiratory status (and, optionally, a dyspnea status) of the user is determined as described above with reference to steps 11-16 of FIG. 2. However; the method of this embodiment additionally involves the performance of steps 50-58 as shown in FIG. 5.

In step 50 first waking activity measurements of the user are obtained during a third test period, for example using additional sensor 25. In embodiments where additional sensor 25 is an activity actigraph, the user obtains these measurements by wearing the activity actigraph for the duration of the third test period, whilst they perform their normal daily routine. Preferably the length of the third test period is at least three hours. Preferably the third test period covers the morning, afternoon and evening of a given day. In some embodiments the third test period is adjacent to, overlaps with, or encompasses the first test period (during which first video data is obtained). In preferred embodiments the third test period encompasses the first test period (i.e. the user obtains first video data during the time when their activity is being measured by additional sensor 25). In some embodiments the third test period comprises a section of the time for which the additional sensor 25 was activated. In some embodiments the third test period comprises a plurality of separated time periods. For example, in one specific embodiment, the third test period comprises a one hour period in the morning, a one hour period in the afternoon, and a one hour period in the evening. In some embodiments the system requests the user (e.g. by means of a message displayed by the portable electronic device or a reminder sent by SMS or e-mail) to obtain first video data and first waking activity data daily, whilst leaving the user free to choose the exact time each day at which to acquire each type of data. In preferred embodiments, if the user has not obtained a particular kind of data by a certain time (6 pm, say, if daily tests are required) a reminder is generated, for example by the portable electronic device 20, and displayed or sent to the user.

In step 51 second waking activity measurements of the user during a fourth test period are obtained, in the same manner as the first waking activity measurements. Preferably the fourth test period is the same or similar with respect to length and other features (e.g. the number of separate time periods it comprises) as the third test period. Step 51 is performed after step 50, such that the third and fourth test periods are spaced apart in time. Preferably the time between the third and fourth test periods is related to the time between the first and second test periods (during which first video data is obtained). For example, in some embodiments video data and waking activity data are both obtained once per day (a single test period is considered as one acquisition of data, even if that test period comprises a plurality of separate time periods). As with the video data, it will be appreciated that the precise length of time which elapses between the performance of steps 50 and 51 is not crucial to the functioning of the invention. Indeed, in preferred embodiments the user may vary the times at which they obtain the first and second waking activity data to enable them to perform these steps at times which are convenient for them. In some embodiments the user is requested (for example by means of a message generated by the portable electronic device 20) to perform steps 50 and/or 51 within a specified time window. Preferably the times at which step 50 and step 51 are performed are recorded, for example by the processing unit of the portable electronic device 20. As with the video data, it will be appreciated that steps 50 and 51 need not represent consecutive acquisitions of waking activity data by the user. For example, in some embodiments the user obtains waking activity data once every day but the first and second waking activity data is spaced apart by more than one day.

In step 52 first sleep motion measurements of the user are obtained during a fifth test period, for example using additional sensor 24. In embodiments where additional sensor 24 is a sleep actigraph, the user obtains these measurements by wearing the sleep actigraph whilst they are asleep, for at least the duration of the fifth test period. The user should activate the additional sensor 24 once they are in bed, and should deactivate it when they wake up. In preferred embodiments the fifth test period is a section of the time for which the additional sensor 24 was activated. In some embodiments a particular section of the time for which the additional sensor 24 was activated is selected, e.g. by the processing unit of the portable electronic device, to be the fifth test period. In such embodiments the selection may be based on indications in the sleep motion data that the patient was actually asleep during the selected period. Preferably the length of the fifth test period is at least several hours. In preferred embodiments the fifth test period occurs close in time to the first test period (i.e. preferably during the night before or after the day in which the first test period occurs).

In step 53 second sleep motion measurements of the user during a sixth test period are obtained, in the same manner as the first sleep motion measurements. Preferably the sixth test period is the same length as the fifth test period. Step 53 is performed after step 52, such that the fifth and sixth test periods are spaced apart in time. Preferably the time between the fifth and sixth test periods is related to the time between the first and second test periods (during which first video data is obtained). In preferred embodiments video data and sleep motion data are both obtained once per 24 hours. As with the video data and waking activity data, it will be appreciated that the precise length of time which elapses between the fifth and sixth test periods is not crucial to the functioning of the invention. Indeed, since the user may not always fall asleep and/or wake-up at the same time each day, it will often be necessary to select fifth and sixth test periods which occur at different times of night. Preferably the start and/or end times of the fifth and sixth test periods are recorded, for example by the processing unit of the portable electronic device 20. As with the video data and the waking activity data, it will be appreciated that steps 52 and 53 need not represent consecutive acquisitions of sleep motion data by the user.

It will be appreciated that the order of steps 50-53 may differ from that shown in FIG. 5. For example, in preferred embodiments steps 50 and 52 are performed before steps 51 and 53.

In step 54 the first and second waking activity measurements are analyzed to detect trends in the waking activity levels of the user. A waking activity level is calculated for each set of waking activity measurements (a set of waking activity measurements being those obtained during a particular daytime period). These levels are compared to each other to identify any trends.

In embodiments in which waking activity measurements have been obtained in the time between the acquisition of the first waking activity measurements and the second waking activity measurements, then these intermediate waking activity measurements are also used in the analysis. In preferred embodiments waking activity measurements are obtained daily, but the first and second waking activity measurements are separated in time by one week. Clearly, in such embodiments seven sets of waking activity measurements are used in the analysis. In preferred embodiments step 54 is performed with the same frequency as the acquisition of new waking activity measurements (although it will be appreciated that this step could be performed less frequently).

In step 55 the first and second sleep motion measurements are analyzed to detect trends in the sleep quality of the user. The sleep motion measurements are analyzed to detect waking events. In some embodiments the frequency of the detected waking events is calculated. In some embodiments the duration of the detected waking events is calculated. The results of the detecting and/or calculating are used to determine a sleep quality level corresponding to each of the first and second sleep motion measurements. In ideal situation no waking events occur, which indicates an adequate sleep quality. Increases in the frequency and/or duration of waking events indicate a worsening in sleep quality. A sleep quality level is calculated for each set of sleep motion measurements (a set of sleep motion measurements being those obtained during a particular night-time period). These levels are compared to each other to identify any trends.

In embodiments in which sleep motion measurements have been obtained in the time between the acquisition of the first sleep motion measurements and the second sleep motion measurements, then these intermediate sleep motion measurements are also used in the analysis. In preferred embodiments sleep motion measurements are obtained nightly, but the first and second sleep motion measurements are separated in time by one week. In preferred embodiments step 55 is performed with the same frequency as the acquisition of new sleep motion measurements (although it will be appreciated that this step could be performed less frequently).

In step 56 a waking activity status of the user is determined based on the results of step 54. In a specific embodiment, if there is not a sustained decreasing trend in the waking activity levels, then the user is determined to have a low risk (green) waking activity status. If a sustained decreasing trend is identified, but the deterioration over the analysis period is less than a predefined threshold, the user is determined to have a medium risk (yellow) waking activity status. If a sustained decreasing trend is identified, and the deterioration over the analysis period is greater than or equal to the predefined threshold, the user is determined to have a high risk (red) waking activity status.

In step 57 a sleep quality status of the user is determined based on the results of step 55. In a specific embodiment, if there is not a sustained decreasing trend in the sleep quality levels, then the user is determined to have a low risk (green) sleep quality status. If a sustained decreasing trend is identified, but the deterioration over the analysis period is less than a predefined threshold, the user is determined to have a medium risk (yellow) sleep quality status. If a sustained decreasing trend is identified, and the deterioration over the analysis period is greater than or equal to the predefined threshold, the user is determined to have a high risk (red) sleep quality status.

Step 58 replaces step 17 of FIG. 2. In this step, a signal is output, for example by the processing unit of the portable electronic device 20, based on the current respiratory status of the user (as determined in step 16 of FIG. 2), the current waking activity status of the user (as determined in step 56), and on the current sleep quality status of the user (as determined in step 57). The signal output at step 58 is as discussed above in relation to step 17 of FIG. 2, except that it also contains information about the current waking activity status and the current sleep quality status of the user.

It will be appreciated that embodiments are possible in which the system comprises only the first additional sensor 24, or in which only the first additional sensor 24 is used, so that only sleep motion data is measured (alongside the acquisition of video data). In such embodiments steps 50, 51, 54 and 56 are omitted from the method of FIG. 5, and in step 58 the signal is output based on just the determined respiratory status and the determined sleep quality status.

Alternatively, embodiments are possible in which the system comprises only the second additional sensor 25, or in which only the second additional sensor 25 is used, so that only waking activity data is measured (alongside the acquisition of video data). In such embodiments steps 52, 53, 55 and 57 are omitted from the method of FIG. 5, and in step 58 the signal is output based on just the determined respiratory status and the determined waking activity status.

There is therefore provided a method, apparatus and system that allow the status of a user with a pulmonary condition to be monitored so as to detect and/or predict a worsening of their condition using only a portable electronic device.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method for non-invasively monitoring a health status of a user with a pulmonary condition, the method comprising:
(a) providing a processing unit;
(b) obtaining first video data of the user during a first test period;
(c) analyzing the obtained first video data to determine a first respiratory signal for the user;
(d) detecting any cough events which are present in the first respiratory signal, said cough events being detected from peaks of the first respiratory signal;
(e) obtaining second video data of the user during a second, later, test period;
(f) analyzing the obtained second video data to determine a second respiratory signal for the user;
(g) detecting any cough events which are present in the second respiratory signal, said cough events being detected from peaks of the second respiratory signal;
(h) determining a respiratory status of the user based on the result of the detecting in steps (d) and (g); and
(i) outputting a signal containing information about the respiratory status of the user, wherein at least steps (c), (d) and (f)-(i) are performed by the provided processing unit.

2. The method of claim 1, wherein the first respiratory signal for the user is extracted from the first video data by analyzing motion vectors in said first video data, and where the second respiratory signal for the user is extracted from the second video data by analyzing motion vectors in said second video data.

3. The method of claims 1, wherein step (d) comprises determining the number of cough events present in the first respiratory signal and step (g) comprises determining the number of cough events present in the second respiratory signal, and wherein step (h) comprises:
comparing the results of steps (d) and (g) with an upper threshold and a lower threshold; and
if the number of detected cough events during the predefined time frame is less than the lower threshold, determining the respiratory status of the user as a first risk level;
if the number of detected cough events during the predefined time frame is greater than or equal to the lower threshold but less than the upper threshold, determining the respiraory status of the user as a second risk level; and
if the number of detected cough events during the predefined time frame is greater than or equal to the upper threshold, determining the respiratory status of the user as a third risk level.

4. The method of claim 1, further comprising detecting and analyzing features associated with dyspnea in the first respiratory signal and in the second respiratory signal.

5. The method of claim 4, wherein the step of detecting and analyzing features associated with dyspnea in the first respiratory signal and in the second respiratory signal comprises:
calculating values of the mean respiration rate and mean respiration amplitude for each respiratory signal;
comparing the calculated mean respiration rate values to one or more predefined thresholds; and
analyzing the calculated mean respiration rate values and mean respiration amplitude values to identify trends in the mean respiration rate and mean respiration amplitude.

6. The method of claim 4, wherein the respiratory status is determined based additionally on the results of the detecting and analyzing of features associated with dyspnea.

7. The method of claim 1, further comprising sending or displaying a reminder message to the user, if the first and/or second video data has not been obtained by a predefined time.

8. The method of claim 1, wherein the signal output in step (i) is arranged to cause a message containing the information contained in the signal to be sent to an electronic device associated with a caregiver.

9. The method of claim 1, further comprising:
obtaining first waking activity measurements of the activity of the user during a third test period;
obtaining second waking activity measurements of the activity of the user during a fourth test period;
analyzing the first and second waking activity measurements to detect trends in the waking activity levels of the user; and
determining a waking activity status of the user based on the detected waking activity trends;
wherein the signal output in step (i) additionally contains information about the waking activity status of the user.

10. The method of claim 1, further comprising:
obtaining first sleep motion measurements of the activity of the user during a fifth test period;
obtaining second sleep motion measurements of the activity of the user during a sixth test period;
analyzing the first and second sleep motion measurements to detect trends in the sleep quality of the user; and
determining a sleep quality status of the user based on the detected sleep quality trends;
wherein the signal output in step (i) additionally contains information about the sleep quality status of the user.

11. A portable device for non-invasively monitoring a health status of a user with a pulmonary condition, the device comprising:
a processing unit having a camera input for receiving video data of the user obtained by a camera; wherein the processing unit is configured to perform at least steps (c), (d) and (f)-(i) of the method of claim 1.

12. The portable device of claim 11, wherein the processing unit is further configured for determining a dyspnea status of the user based on the results of the detecting and analyzing of features associated with dyspnea, wherein the signal output in step (i) additionally contains information about the dyspnea status of the user.

13. The portable device of claim 12, wherein the processing unit is further configured for determining mean respiration rate values which exceed the one or more predefined thresholds to be indications of dyspnea, wherein determining a dyspnea status of the user comprises:
if none of the calculated mean respiration rate values are determined to be indications of dyspnea, and no sustained trends of decreasing respiration amplitude and/or increasing respiration rate are identified, determining the dyspnea status of the user as a first risk level;
if none of the calculated mean respiration rate values are determined to be indications of dyspnea and at least one sustained trend of decreasing respiration amplitude and/or increasing respiration rate is identified, determining the dyspnea status of the user as a second risk level; and
if the number of mean respiration rate values which are determined to be indications of dyspnea during a predefined time frame is greater than or equal to a predefined dyspnea indication threshold, determining the dyspnea status of the user as a third risk level.

14. A system for non-invasively monitoring a health status of a user with a pulmonary condition, the system comprising:
a portable device according to claim 11, configured to receive activity measurements from a sensor; and
one or more sensors for measuring activity of the user, configured to send activity measurements to the portable device;
wherein the processing unit is configured to perform the method, wherein the steps of obtaining sleep motion measurements and obtaining waking activity measurements comprise receiving activity measurements from the one or more sensors.

15. The system of claim 14, wherein the one or more sensors comprise an accelerometer or a gyroscope and/or an activity actigraph and/or a sleep actigraph.

16. (canceled)

17. A computer program product, comprising computer readable code embodied therein, the computer readable code being configured such that, on execution by a suitable computer or processing unit, the computer or processing unit performs the method described in claim 1, except step (a).
